(19) United States
(12) Patent Application Publication
(10) Pub. No.: US 2020/0367967 A1
(43) Pub. Date: Nov. 26, 2020

(54) MICROLENS ARRAY, AND LASER BEAM HAND PIECE AND THERAPEUTIC LASER DEVICE COMPRISING SAME

(71) Applicant: LUTRONIC CORPORATION, Goyang-si, Gyeonggi-do (KR)

(72) Inventor: Hee Chul LEE, Goyang-si, Gyeonggi-do (KR)

(21) Appl. No.: 16/769,436

(22) PCT Filed: Nov. 15, 2018
(86) PCT No.: PCT/KR2018/013959

§ 371 Date: Jun. 3, 2020

(30) Foreign Application Priority Data
Dec. 5, 2017 (KR) 10-2017-0166195

(51) Int. Cl. A61B 18/20; G02B 3/02

(52) U.S. Cl. CPC A61B 18/20; G02B 3/0006; G02B 3/02

(57) ABSTRACT

A microlens array for converting incident light into a multi-spot beam to focus an object, the microlens array having a plurality of microlenses each having a curved surface forming a predetermined focal length, wherein at least some of the plurality of microlenses have different focal lengths or are different in the height position from which the corresponding curved surface starts.

### CROSS REFERENCE TO RELATED APPLICATIONS

This application is a National Stage of International Application No. PCT/KR2018/013959, having an International Filing Date of 15 Nov. 2018, which designated the United States of America, and which International Application was published under PCT Article 21(2) as WO Publication No. 2019/112199 A1, which claims priority from and the benefit of Korean Patent Application No. 10-2017-0166195, filed on 5 Dec. 2017, the disclosures of which are incorporated herein by reference in their entireties.

### BACKGROUND

### 1. Field

Aspects of the present disclosure relate to a microlens array, and a laser beam hand piece and a laser therapeutic device comprising the same.

### 2. Brief Description of Related Developments

Laser beams are currently used in various fields for industrial, medical, or military purposes. Especially, medical lasers are coming into widespread use in the fields of surgery, internal medicine, ophthalmology, dermatology, dentistry, and so on, because predetermined energy of laser beams can be locally focused and noninvasive treatment is permitted.

### SUMMARY

Provided is a microlens array which is employed in a laser therapeutic device and is capable of irradiating a multi-spot beam with improved uniformity onto the skin.

According to an aspect, provided is a microlens array for converting incident light into a multi-spot beam to focus an object, the microlens array comprising: a plurality of microlenses each having a curved surface forming a predetermined focal length, wherein at least some of the plurality of microlenses have different focal lengths or are different in the height position from which the corresponding curved surface starts.

The shapes and arrangement of the plurality of microlenses may be determined such that a beam spot trajectory formed by the microlens array forms a non-planar surface.

The non-planar surface may be convex facing the microlens array.

The focal lengths of the plurality of microlenses may be all the same, and two or more of the plurality of microlenses may be different in the height position from which the corresponding curved surface starts.

The shapes and arrangement of the plurality of microlenses may be determined such that the height positions gradually increase away from the center toward the periphery of the microlens array.

Alternatively, the height positions of the plurality of microlenses, from which the corresponding curved surfaces start, may be all the same, and two or more of the plurality of microlenses may have different focal lengths.

The shapes and arrangement of the plurality of microlenses may be determined such that the respective focal lengths of the plurality of microlenses gradually increase away from the center toward the periphery of the microlens array.

The two or more microlenses may be made of the same refractive index material and may have different curved surface shapes.

Alternatively, the two or more microlenses may have the same curved surface shape and may be made of different refractive index materials.

According to a mode of the present disclosure, provided is a method of designing a microlens array for converting incident light into a multi-spot beam and to focus an object, wherein the shapes and arrangement of the plurality of microlenses are determined such that beam spots formed by the respective microlenses are positioned at a uniform depth within the object.

According to a mode of the present disclosure, provided is a laser beam hand piece comprising; a lens unit adjusting a spot size of an incident laser beam; and a microlens array for converting the incident laser beam having a spot size adjusted in the lens unit into a multi-spot beam, and focusing the same on an object, the microlens array including a plurality of microlenses each having a curved surface forming a predetermined focal length, wherein at least some of the plurality of microlenses have different focal lengths or are different in the height position from which the corresponding curved surface starts.

The shapes and arrangement of the plurality of microlenses may be determined such that a beam spot trajectory formed by the microlens array forms a non-planar surface.

The non-planar surface may have a convex surface facing the microlens array.

The shapes and arrangement of the plurality of microlenses may be determined such that the height positions from which the corresponding curved surfaces start gradually increase away from the center toward the periphery of the microlens array.

Alternatively, the shapes and arrangement of the plurality of microlenses may be determined such that the focal distances of the plurality of microlenses gradually increase away from the center toward the periphery of the microlens array.

According to a mode of the present disclosure, provided is a laser therapeutic device including a laser generator; a guide arm guiding the laser output from the laser generator; a lens unit adjusting the spot size of an incident laser beam; and a microlens array for converting the incident laser beam having a spot size adjusted in the lens unit into a multi-spot beam, and focusing an object, the microlens array including a plurality of microlenses each having a curved surface forming a predetermined focal length, wherein at least some of the plurality of microlenses have different focal lengths or are different in the height position from which the corresponding curved surface starts.

The shapes and arrangement of the plurality of microlenses may be determined such that a beam spot trajectory formed by the microlens array forms a non-planar surface.

The non-planar surface may have a convex surface facing the microlens array.

The shapes and arrangement of the plurality of microlenses may be determined such that the height positions from which the corresponding curved surfaces start gradually increase away from the center toward the periphery of the microlens array.

Alternatively, the shapes and arrangement of the plurality of microlenses may be determined such that the focal distances of the plurality of microlenses gradually increase away from the center toward the periphery of the microlens array.

The microlens array may convert an incident beam into a multi-spot beam and then irradiate the same into the uneven skin, and thus may improve uniformity in the depth position within the skin, where the multi-spot beam is formed.

The microlens array may be employed in the laser therapeutic device, and thus can be expected to have uniform skin therapeutic effect.

### BRIEF DESCRIPTION OF THE DRAWINGS

FIG. 1 is a plan view illustrating a schematic configuration of a microlens array according to an aspect.

FIG. 2 is a cross-section view taken along the line A-A′, illustrating the microlens array of FIG. 1 with a beam spot trajectory formed by the microlens array.

FIG. 3 is a cross-section view illustrating a schematic configuration of a microlens array according to a comparative example with a beam spot trajectory formed by the microlens array.

FIG. 4 is a plan view illustrating a schematic configuration of a microlens array according to another aspect.

FIG. 5 is a cross-section view taken along the line B-B′, illustrating the microlens array of FIG. 4 with a beam spot trajectory formed by the microlens array.

FIG. 6 is a plan view illustrating a schematic configuration of a microlens array according to still another aspect.

FIG. 7 is a schematic perspective view illustrating a configuration of a laser therapeutic device according to an aspect.

FIG. 8 is an exploded perspective view concretely illustrating a laser beam hand piece provided in the laser therapeutic device shown in FIG. 7.

FIG. 9 illustrates that the skin surface is projected by a tip of the laser beam hand piece in a process of treating the skin using the laser therapeutic device shown in FIG. 7.

### DETAILED DESCRIPTION

The present disclosure allows for various changes and numerous aspects, and specific aspects will now be illustrated in the drawings and described in detail in the detailed description. Advantages and features of the present invention and a method for achieving them will be apparent with reference to aspects of the disclosure described below together with the attached drawings. However, the present disclosure is not limited to the disclosed aspects, but may be implemented in various manners.

Hereinafter, aspects of the present disclosure will be described in greater detail with reference to the accompanying drawings. In describing the aspects with reference to drawings, an identical or corresponding element will be referred to as an identical reference numeral, and repeated descriptions will not be given.

In the following aspects, the terms first, second, etc. are not intended to be limiting but are only used to distinguish one element component, from another.

In the following aspects, the singular forms are intended to include the plural forms, unless the context clearly indicates otherwise.

In the following aspects, the terms “comprises” and/or “comprising,” when used in this specification, specify the presence of stated features, and/or components, but do not preclude the presence or addition of one or more other features, and/or components.

In the following aspects, when a part such as a region or a component is referred to as being “on” another part, it can be directly on the other part or intervening regions or components may be present.

In the drawings, for the sake of convenient explanation, the size of each component will be exaggerated or reduced. For example, for brevity and clarity, the size and thickness of each component appearing on each drawing are shown in an arbitrary manner, and the present disclosure is not so limited.

When a certain aspect may be implemented otherwise, a particular process may be performed in a different order than described herein. For example, two processes described in succession may, in fact, be executed substantially concurrently or may sometimes be executed in the reverse order than described herein.

In the following aspects, when regions or components are referred to as being connected to each other, they can be directly connected to each other or can be indirectly connected to each other due to other intervening regions or components being present there between.

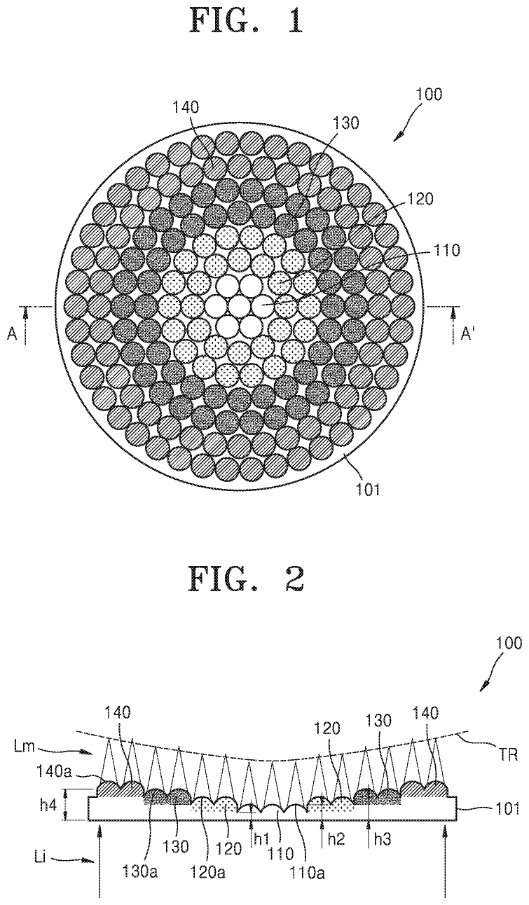

FIG. 1 is a plan view illustrating a schematic configuration of a microlens array according to an aspect, and FIG. 2 is a cross-section view taken along the line A-A′, illustrating the microlens array of FIG. 1 with a beam spot trajectory formed by the microlens array.

The microlens array 100 converts an incident light into a multi-spot beam to focus the same on an object and includes a plurality of microlenses 110, 120, 130 and 140 each having a curved surface forming a predetermined focal length.

The microlens array 100 may be formed by processing one plane of a transparent base 101 so as to have a plurality of predetermined curved surfaces. The transparent base 101 may be made of glass or a transparent plastic material, and the focal distance of each of the plurality of microlenses 110, 120, 130 and 140 is determined according to the refractive index of the transparent base 101 and the shape of the curved surface of each of the plurality of microlenses 110, 120, 130 and 140. The curved surface of each of the plurality of microlenses 110, 120, 130 and 140 may be spherical or aspherical.

The microlens array 100 according to one or more embodiments may include a plurality of microlenses 110, 120, 130 and 140 designed such that the trajectory TR of beam spots formed by the microlens array 100 forms a non-planar surface. In other words, the shapes and positions in the array of the plurality of microlenses 110, 120, 130 and 140 may be determined so as to make the trajectory TR of beam spots become a non-planar surface.

Designing the microlens array 100 in such a manner is to make beam spots formed by the plurality of microlenses 110, 120, 130 and 140 positioned at a uniform depth within an object, considering that the skin surface which is the object for beam irradiation of device employing the microlens array 100, e.g., a laser therapeutic device is generally non-planar. In order to make the beam spot trajectory TR similar to the surface shape of the object, at least some of the plurality of microlenses 110, 120, 130 and 140 may have different focal lengths or may be different in the height position from which the corresponding curved surface starts.

In the microlens array 100 according to one or more embodiments, the shapes and arrangement of the plurality of microlenses 110, 120, 130 and 140 may be determined such that the height positions from which the corresponding curved surface starts gradually increase away from the center toward the periphery of the microlens array 100.

Referring to FIG. 2, the microlens array 100 according to one or more aspects may include first to fourth microlenses 110, 120, 130 and 140 having the same focal lengths and being different in the height position from which the corresponding curved surface starts. The height positions from which the respective curved surfaces of the first to fourth microlenses 110, 120, 130 and 140 start may be differently set according to the positions thereof arranged in the microlens array.

Each of the first microlenses 110 may have a height position h1 from which a curved surface 110a starts, each of the second microlenses 120 may have a height position h2 from which a curved surface 120a starts, each of the third microlenses 130 may have a height position h3 from which a curved surface 130a starts, and each of the fourth microlenses 140 may have a height position h4 from which a curved surface 140a starts, and the relationship h1<h2<h3<h4 may be established. The first microlenses 110 may be disposed at the center of the microlens array 100, the second microlenses 120 may enclose the first microlenses 110, the third microlenses 130 may enclose the second microlenses 120, and the fourth microlenses 140 may enclose the third microlenses 130. The first to fourth microlenses 110, 120, 130 and 140 shown in the illustrated embodiment are radially symmetrical positioned, but aspects are not limited thereto. Rather, the first to fourth microlenses 110, 120, 130 and 140 may be asymmetrically arranged. In other words, the first microlenses 110 circularly arranged and the second microlenses 120 circularly arranged so as to enclose the first microlenses 110 are shown in the illustrated aspect, but aspects are not limited thereto. The second microlenses 120 may elliptically enclose the first microlenses 110 or may enclose the first microlenses 110 in any other shape. Specific numerals for the respective heights of the curved surface 110a, 120a, 130a and 140a of the first to fourth microlenses 110, 120, 130 and 140 start, and numbers of the first to fourth microlenses 110, 120, 130 and 140 may be determined in consideration of the focal lengths of the first to fourth microlenses 110, 120, 130 and 140, the diameters of the first to fourth microlenses 110, 120, 130 and 140, or the shape of the object. For example, the diameter of each of the first to fourth microlenses 110, 120, 130 and 140 may be 0.8 mm, and h1, h2, h3 and h4 may be 0.2 mm, 0.4 mm, 0.6 mm and 0.8 mm, respectively, but aspects are not limited thereto. In addition, four types of microlenses having different curved surface height positions are illustrated, but aspects are not limited thereto. The microlenses may be varied as another number of types of microlenses.

The microlens array 100 converts incident light (Li) into a multi-spot beam (Lm). Here, the first to fourth microlenses 110, 120, 130 and 140 are shaped to have the same focal length, but are different in height position from which each of the curved surfaces 110a, 120a, 130a and 140a starts, and thus the beam spot trajectory TR formed thereby forms a non-planar surface. The trajectory TR may be, for example, convex facing the microlens array 100. However, the illustrated shape is provided only by way example, and kinds, arrays or shapes of the microlenses 110, 120, 130 and 140 provided in the microlens array 100 may vary in consideration of the shape of a desired trajectory TR.

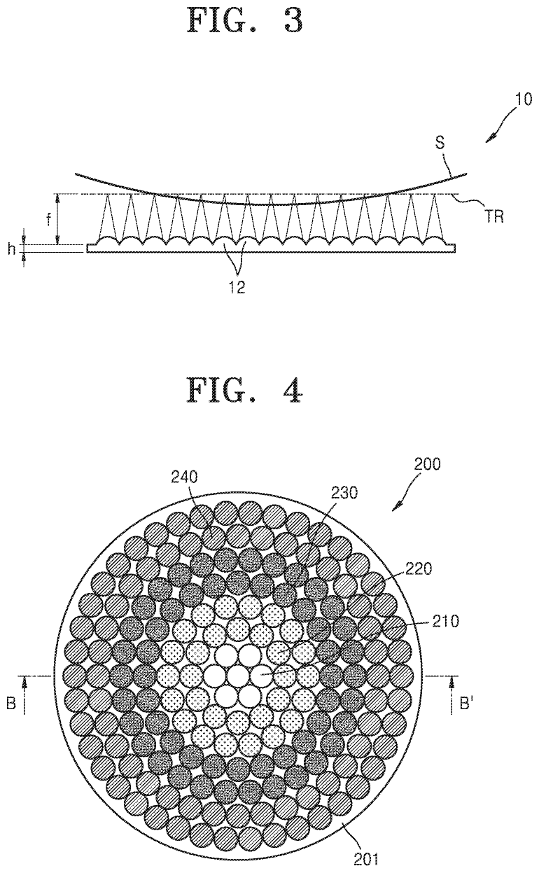

FIG. 3 is a cross-section view illustrating a schematic configuration of a microlens array 10 according to a comparative example with a beam spot trajectory formed by the microlens array 10.

The microlens array 10 according to a comparative example include a plurality of microlenses 12 which are all the same. That is to say, the plurality of microlenses 12 have the same focal length (f) and are the same in the height position (h) from which the curved surface 12a starts. Therefore, the beam spot trajectory TR formed by the microlens array 10 may become a plane.

As described above, when the microlenses 12 provided in the microlens array 10 have the same shape irrespective of the positions in the array, the beam spot trajectory TR formed by the microlens array 10 forms a plane, by which, when a laser therapeutic device employing the microlens array 10 irradiates a laser beam on the skin S, which is generally non-planar, the laser beam is irradiated to different depths on various positions of the skin S.

Generally, the depths within the skin onto which the laser beam is irradiated are differently set according to the purpose of treatment. For example, there may be a case that a laser beam is irradiated to the epidermis of 0.1 mm from the skin surface or to the dermis of 1 to 4 mm in depth. However, when the beam spot trajectory forms a plane, uniformity in the depth position may not be achieved. For example, beam spots may be formed at some positions deeper than intended, or there may be some regions that the beam spots may not well reach. In such cases, it may be difficult to achieve a desired therapeutic effect.

By contrast, the microlens array 10 according to one or more aspects includes microlenses designed by maximally reflecting the shape of the skin surface, thereby improving the uniformity in the depth position where each beam spot is formed, and ultimately increasing the therapeutic effect.

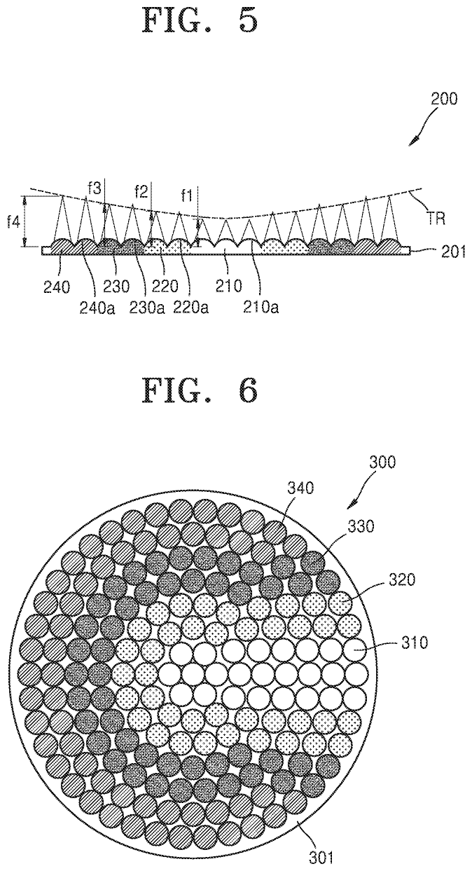

FIG. 4 is a plan view illustrating a schematic configuration of a microlens array according to another aspect, and FIG. 5 is a cross-section view taken along the line B-B′, illustrating the microlens array of FIG. 4 with a beam spot trajectory formed by the microlens array.

The microlens array 200 includes first to fourth microlenses 210, 220, 230 and 240 having the same height positions from which curved surfaces 210a, 220a, 230a and 240a start and different focal lengths. The microlens array 200 may be formed by processing one plane of a transparent base 201 so as to have a plurality of predetermined curved surfaces 210a, 220a, 230a and 240a, and shapes and arrangement of the first to fourth microlenses 210, 220, 230 and 240 may be determined such that the focal lengths gradually increase away from the center toward the periphery of the microlens array 200.

Each of the first microlenses 210 may have a focal length f1, each of the second microlenses 220 may have a focal length f2, each of the third microlenses 230 may have a focal length f3, and each of the fourth microlenses 240 may have a focal length f4, and the relationship f1<f2<f3<f4 may be established. The first microlenses 210 may be disposed at the center of the microlens array 200, the second microlenses 220 may enclose the first microlenses 210, the third microlenses 230 may enclose the second microlenses 220, and the fourth microlenses 240 may enclose the third microlenses 230. The first to fourth microlenses 210, 220, 230 and 240 shown in the illustrated aspect are radially symmetrical positioned, but aspects are not limited thereto. Rather, the first to fourth microlenses 210, 220, 230 and 240 may be asymmetrically arranged. The focal lengths f1, f2, f3 and f4 of the first to fourth microlenses 210, 220, 230 and 240 and numbers of the first to fourth microlenses 210, 220, 230 and 240 may be determined in consideration of the diameters of the first to fourth microlenses 210, 220, 230 and 240, or the shape of the object.

In order to make focal lengths different from one another, the first to fourth microlenses 210, 220, 230 and 240 may be made of the same material, and the respective curved surfaces 210a, 220a, 230a and 240a may have different shapes from one another. Alternatively, the first to fourth microlenses 210, 220, 230 and 240 may have the same curved surface shape and may be made of different refractive index materials.

FIG. 6 is a plan view illustrating a schematic configuration of a microlens array according to still another aspect.

The microlens array 300 includes first to fourth microlenses 310, 320, 330 and 340 having different focal lengths or positions from which corresponding curved surfaces start so as to form a non-planar beam spot trajectory.

The microlens array 300 according to one or more aspects is different from the microlens array 100 (200) with respect to the distribution of the first to fourth microlenses 310, 320, 330 and 340. The second microlenses 320 may be arranged to partially enclose some of the first microlenses 310, rather than to entirely enclose the first microlenses 310. For example, the second microlenses 320 may enclose the first microlenses 310 in a horseshoe shape, the third microlenses 330 may enclose the second microlenses 320 in a horseshoe shape, and the fourth microlenses 340 may enclose the third microlenses 330 in a horseshoe shape.

The first to fourth microlenses 310, 320, 330 and 340 for forming a non-planar trajectory may be different in height positions from which corresponding curved surfaces start, as shown in FIG. 2. Alternatively, the first to fourth microlenses 310, 320, 330 and 340 may be provided to have different focal lengths.

The above-described microlens arrays 100, 200 and 300 are provided as specific implementation examples of multi-beam spots forming non-planar trajectories, but aspects are not limited thereto. Any one of microlens arrays 100, 200 and 300, a combination thereof or a modification thereof may be implemented. For example, a microlens array including a plurality of microlenses having all the same focal length and some of the plurality of microlenses being different in the height position from which the corresponding curved surface starts, or a microlens array including a plurality of microlenses being all the same in the height position from which the corresponding curved surface starts and some of the plurality of microlenses having different focal lengths, may be implemented. In addition to these implementation types, a combination thereof, that is, a microlens array including some of a plurality of microlenses having different focal lengths and some other of the plurality of microlenses being different in the curved surface height position, may also be implemented.

The microlens arrays 100, 200 and 300 may be employed in a laser therapeutic device, and is capable of irradiating a multi-spot beam with improved uniformity into the uneven skin, and thus may improve uniformity in the depth position where the multi-spot beam is formed in treating the skin.

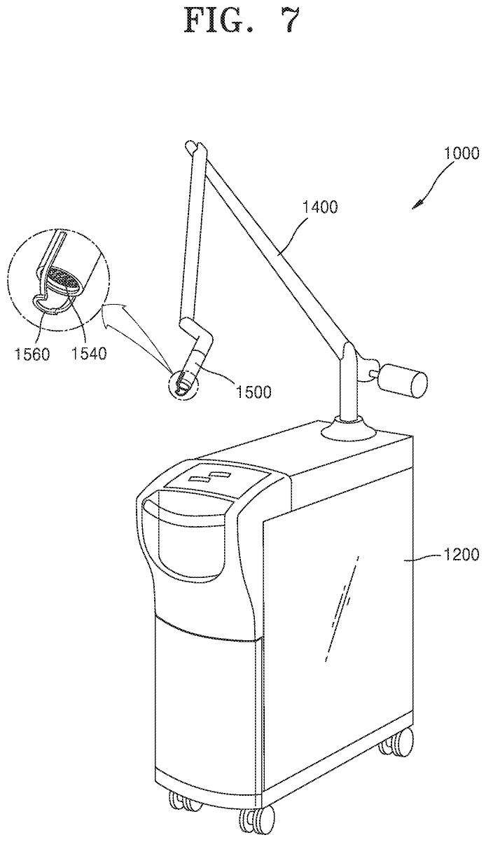

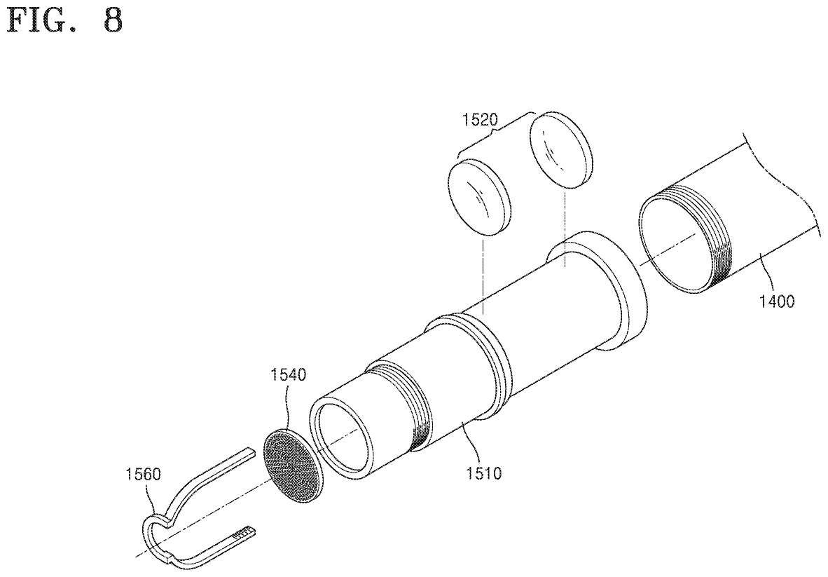

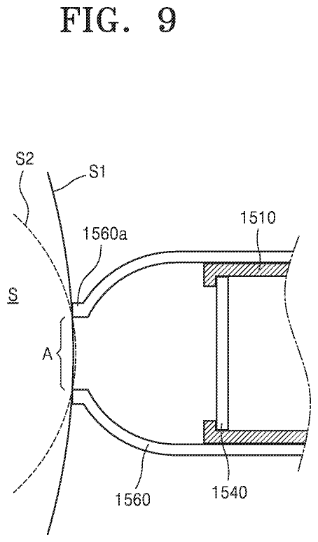

FIG. 7 is a schematic perspective view illustrating a configuration of a laser therapeutic device according to an aspect, FIG. 8 is an exploded perspective view concretely illustrating a laser beam hand piece provided in the laser therapeutic device shown in FIG. 7, and FIG. 9 illustrates that the skin surface is projected by a tip of the laser beam hand piece in a process of treating the skin using the laser therapeutic device shown in FIG. 7.

The laser therapeutic device 1000 includes a laser generator 1200, a guide arm 1400 guiding a laser beam output from the laser generator 1200, and a laser beam hand piece 1500 adjusting the spot size of the laser beam guided by the guide arm 1400, converting the same into a multi-spot beam and focusing the same on an object.

The laser generator 1200 generates a laser of a predetermined wavelength band for treatment, and the guide arm 1400 guides the generated laser.

Referring to FIG. 8, the laser beam hand piece 1500 includes a lens unit 1520 adjusting the spot size of the laser beam guided by the guide arm 1400, and a microlens array 1540 converting the beam having a spot size adjusted by the lens unit 1520 into the multi-spot beam and focusing the same on the object. The lens unit 1520 consisting of two sheets of lenses is shown in the illustrated aspect, but is provided only by way of example. The microlens array 1540 includes a plurality of microlenses each having a curved surface forming a predetermined focal length, and the shapes and arrangement of the plurality of microlenses are so set that a beam spot trajectory formed by the microlens array 1540 forms a non-planar surface. At least some of the plurality of microlenses constituting the microlens array 1540 may have different focal lengths and may be different in the height position from which the corresponding curved surface starts. Any one of the microlens arrays 100, 200 and 300, a combination thereof, or a modified array thereof may be employed as the microlens array 1540.

The laser beam hand piece 1500 may further include a barrel 1510 fixedly accommodating the lens unit 1520 and the microlens array 1540, and a tip 1560 making contact with an object, for example, the skin to be treated, may be formed at an end of the laser beam hand piece 1500. A sensor may also be provided at an end portion 1560a of the tip 1560 to make a constant pressure applied when the tip 1560 comes into contact with the skin. The illustrated end portion 1560a of the tip 1560 is formed in a horseshoe shape, but is not limited thereto. The end portion 1560a may be formed in a circular shape.

Generally, a trajectory S1 including a treatment object area A of the skin surface S is uneven and non-planar. The laser therapeutic device 1000 according to one or more aspects includes a microlens array 1540 designed to irradiate a multi-spot beam along a non-planar trajectory in consideration of the shape of the treatment object area A, thereby increasing the uniformity in the depth position of the multi-spot beam formed in the treatment object area A.

Meanwhile, when the tip 1560 comes into contact with the skin, the end portion 1560a of the tip 1560 may apply a constant pressure to the skin S. In this case, the trajectory along the surface of the skin S may change from S1 to S2, and the treatment object area A may be more convexly formed than before the tip end portion 1560a comes into contact with the skin. The laser therapeutic device 1000 according to one or more aspects may employ the microlens array 1540 designed to irradiate a multi-spot beam along a non-planar trajectory in consideration of the shape of the treatment object area A or the pressure applied when the tip 1560 comes into contact with the skin. Therefore, the uniformity in the depth position of the multi-spot beam formed in the treatment object area A may be increased.

In the microlens array 1540 provided in the laser therapeutic device 1000, the shapes and arrangement of specific microlenses may be determined in consideration of the shape of the end portion 1560a of the tip 1560 or the pressure expected to be applied when the tip 1560 comes into contact with the skin S. For example, when the end portion 1560a of the tip 1560 is formed in a circular shape, the arrangement shown in FIG. 1 or FIG. 4 may be preferred. When the end portion 1560a of the tip 1560 is formed in a horseshoe shape, the arrangement shown in FIG. 6 may be preferred. However, aspects of the present disclosure are not limited thereto.

While one or more exemplary aspects have been described with reference to the figures, the aspects described herein have been presented by way of example only, and it will be appreciated by those skilled in the art that various changes and other equivalent aspects may be made from the above description. Therefore, the true technical protection scope of the present disclosure should be defined by the inventive concept of the appended claims.

What is claimed is:

1. A microlens array for converting incident light into a multi-spot beam to focus an object, the microlens array comprising:
a plurality of microlenses each having a curved surface forming a predetermined focal length,
wherein at least some of the plurality of microlenses have different focal lengths or are different in the height position from which the corresponding curved surface starts.

2. The microlens array of claim 1, wherein the shapes and arrangement of the plurality of microlenses are determined such that a beam spot trajectory formed by the microlens array forms a non-planar surface.

3. The microlens array of claim 2, wherein the non-planar surface is convex facing the microlens array.

4. The microlens array of claim 1, wherein
the plurality of microlenses have all the same focal length, and
two or more of the plurality of microlenses are different in the height position from which the corresponding curved surface starts.

5. The microlens array of claim 4, wherein the shapes and arrangement of the plurality of microlenses are determined such that the height positions gradually increase away from the center toward the periphery of the microlens array.

6. The microlens array of claim 1, wherein
the height positions of the plurality of microlenses, from which the corresponding curved surfaces start, are all the same, and
two or more of the plurality of microlenses have different focal lengths.

7. The microlens array of claim 6, wherein the shapes and arrangement of the plurality of microlenses are determined such that the respective focal lengths of the plurality of microlenses gradually increase away from the center toward the periphery of the microlens array.

8. The microlens array of claim 6, wherein two or more microlenses are made of the same refractive index material and have different curved surface shapes.

9. The microlens array of claim 7, wherein two or more microlenses have the same curved surface shape and are made of different refractive index materials.

10. A method of designing a microlens array for converting incident light into a multi-spot beam to focus an object, wherein the shapes and arrangement of the plurality of microlenses are determined such that beam spots formed by the respective microlenses are positioned at a uniform depth within the object.

11. A laser beam hand piece comprising:
a lens unit adjusting a spot size of an incident laser beam; and
a microlens array for converting the incident laser beam having a spot size adjusted in the lens unit into a multi-spot beam, and focusing an object, the microlens array including a plurality of microlenses each having a curved surface forming a predetermined focal length, wherein at least some of the plurality of microlenses have different focal lengths or are different in the height position from which the corresponding curved surface starts.

12. The laser beam hand piece of claim 11, wherein the shapes and arrangement of the plurality of microlenses are determined such that a beam spot trajectory formed by the microlens array forms a non-planar surface.

13. The laser beam hand piece of claim 12, wherein the non-planar surface has a convex surface facing the microlens array.

14. The laser beam hand piece of claim 11, wherein the shapes and arrangement of the plurality of microlenses are determined such that the height positions gradually increase away from the center toward the periphery of the microlens array.

15. The laser beam hand piece of claim 11, wherein the shapes and arrangement of the plurality of microlenses are determined such that the respective focal lengths of the plurality of microlenses gradually increase away from the center toward the periphery of the microlens array.

16. A laser therapeutic device comprising:
a laser generator;
a guide arm guiding the laser output from the laser generator;
a lens unit adjusting the spot size of an incident laser beam; and
a microlens array for converting the incident laser beam having a spot size adjusted in the lens unit into a multi-spot beam, and focusing an object, the microlens array including a plurality of microlenses each having a curved surface forming a predetermined focal length, wherein at least some of the plurality of microlenses have different focal lengths or are different in the height position from which the corresponding curved surface starts.

17. The laser therapeutic device of claim 16, wherein shapes and arrangement of the plurality of microlenses are determined such that a beam spot trajectory formed by the microlens array forms a non-planar surface.

18. The laser therapeutic device of claim 16, wherein the non-planar surface has a convex surface facing the microlens array.

19. The laser therapeutic device of claim 16, wherein the shapes and arrangement of the plurality of microlenses are determined such that the height positions gradually increase away from the center toward the periphery of the microlens array.

20. The laser therapeutic device of claim 16, wherein the shapes and arrangement of the plurality of microlenses are determined such that the respective focal lengths of the plurality of microlenses gradually increase away from the center toward the periphery of the microlens array.
